(19) United States
(12) Patent Application Publication
(10) Pub. No.: US 2023/0144829 A1
(43) Pub. Date: May 11, 2023

(54) Integrated Digital Surgical System

(71) Applicant: Genesis Medtech (USA) Inc., Saint Louis Park, MN (US)

(72) Inventors: Shan Wan, Plymouth, MN (US); Ning Li, Pittsburgh, PA (US); Yangyang Chang, St Paul, MN (US); Bin Zhao, Ellicott City, MD (US)

(21) Appl. No.: 17/984,313

(22) Filed: Nov. 10, 2022

Related U.S. Application Data
(60) Provisional application No. 63/277,993, filed on Nov. 10, 2021.

(51) Int. Cl. A61B 34/37; A61B 34/10; G16H 30/40; G16H 50/20

(52) U.S. Cl. CPC A61B 34/37; A61B 34/10; G16H 30/40; G16H 50/20; A61B 2017/00199

(57) ABSTRACT

The present invention discloses a surgical integrated assistance system comprising an integrated surgical device having a housing with one or more ports integrated at one side of the housing. The one or more ports are configured for coupling the integrated surgical device with one or more energy instruments. Further, a display control unit is coupled to a display unit and configured to control operating mechanism of each of the one or more energy instruments. Thereafter, an artificial intelligence and machine learning (AI/ML) enabled module integrated within the housing, to automatically optimize multiple parameters of the one or more energy instruments.

### FIELD OF THE DISCLOSURE

The present invention generally relates to a surgical integrated assistance system, and more particularly relates to the surgical integrated assistance system for simplifying and enhancing medical treatments and analysis.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Mechanical tools have been used in a variety of open surgical procedures for centuries. Such tools became the natural extension of surgeons' hand to perform a specific function for treating diseased tissue and organs. There are different types of surgical procedures that are commonly conducted including laparoscopy, endoscopy, arthroscopy, bronchoscopy, gastroscopy etc. Laparoscopy or laparoscopic procedures are used commonly since it is a minimal invasive solution for a wide spectrum of procedures such as cholecystectomy, appendectomy, hernia, and other more complex general/colorectal/GYN/bariatric procedures. Extensive improvement in the mechanical tools have been made to support surgeon's job to perform surgical cutting, dissection, coagulation, tissue manipulation and management. Therefore, energy driven device, such as advanced RF bipolar, and ultrasonic scalpel system, have gain popularity due to this improvement.

Currently, robotic assisted surgery become prevalent in urology and other surgical procedures as the latest addition of the surgical tools. Even with decades of development, the robotic systems still functions as the extension of surgeon's arm. In these systems, a robot arm is used for holding an instrument for performing a surgical procedure, and a control system is separately used for controlling movement of the arm and its instrument, according to user manipulation of a master manipulator. The control system includes a filter in its forward path to attenuate master input commands that may cause instrument tip vibrations, and an inverse filter in a feedback path to the master manipulator configured to compensate for delay introduced by the forward path filter. To enhance control, master command and slave joint observers are also inserted in the control system to estimate slave joint position, velocity and acceleration commands using received slave joint position commands and torque feedbacks, and estimate actual slave joint positions, velocities and accelerations using sensed slave joint positions and commanded slave joint motor torques. However, these systems may provide limited capability or limited information of multiple energy devices over a screen.

Moreover, the robotic system provides the possibility to integrated endoscope with mechanical and energy surgical tools. Through this integration and electronic driving system, it is possible to provide not only individual tools for cutting and coagulation, but it is also enabling of the integration of each tool's data, including procedure imaging, tissue interaction and tool interdependent awareness, to help surgeon in decision making. As data accumulates over time, this helps human surgeons to effectively tackle complex. procedure, make sound decision, and reduce complication intraoperatively. In the conventional non-robotic laparoscopic surgery, the individual tools, such as energy and mechanical tool, does not have capability to integrate data for this advanced function and surgical support. Even the surgical robot is still lack of this effective integration due to the historic individualized tool design philosophy. It only provides a limited integration and intelligence based on the existing tools. And the high capital cost of robotic systems limits its benefits to certain procedures and hospitals.

Given the difference of the prior arts, there is a need for a surgical system integrating with energy system, imaging interface, other data collecting interface to enable data integration, imaging fusion, and critical information exchange.

### SUMMARY OF THE INVENTION

According to one aspect, a surgical integrated assistance system is disclosed. The surgical integrated assistance system comprises an integrated surgical device. The integrated surgical device comprises a housing having one or more ports integrated at one side of the housing. The one or more ports are configured for coupling the integrated surgical device with one or more energy instruments. The integrated surgical device is having at least one external image input port, at least one output port, a power port, and at least one external intelligence module port, on the other side of the housing. Herein, the one or more energy instruments include at least one of bipolar and advanced bipolar shears, monopolar shear, ultrasonic shear, microwave ablation devices, laser ablation devices, laparoscopic devices, robotics control unit, and endoscope. It can be noted that each of the one or more energy instruments are controlled between high voltage and high frequency energy output of the integrated surgical device. Further, the integrated surgical device comprises an imaging scope connection fabricated on the one side of the housing and configured to input at least one optical image or an ultrasound image. In one embodiment, a display unit detachably connected onto the housing and configured to provide a consolidated output related to the one or more energy instruments. The display unit is constructed in a manner to tilt at one or more angles to provide convenience to the operator. It can be noted that the display unit may be wirelessly connected to the housing using Ethernet. Further, a display control unit is disposed on the side of the housing and coupled to the display unit and configured to control operating mechanism of each of the one or more energy instruments. Further, the surgical integrated assistance system comprises an artificial intelligence and machine learning (AI/ML) enabled module integrated within the housing, to automatically optimize multiple parameters of the one or more energy instruments. Herein, the AI/ML enabled module is communicatively coupled to a cloud network for training models of the AI/ML enabled module and to collect real-time information related to the one or more energy instruments.

In one embodiment, the AI/ML enabled module is configured to generate a three-dimensional (3D) reconstruction of the multi organ model using a pre-operation magnetic resonance imaging (MRI) or computed tomography (CT) scan images as reference points. Further, the AI/ML enabled module collects real time inference with live video feed from the Laparoscope to highlight location of diagnosis. In another embodiment, the AI/ML enabled module calculates operation curve of each of the one or more energy instruments to display on the display unit. The curve provides information related to progress of the diagnosis. Further, the AI/ML enabled module displays operation status reminder of the one or more energy instruments for surgeon's reference.

In another embodiment, the AI/ML enabled module is configured to: retrieve real-time streaming video from each of the one or more energy instruments in operation; compute a machine learning inference for lesion localization and surgical navigation; process intra-operation real-time surgical video; and upload and store the surgical video and data related to each of the one or more energy instruments, employed in an operation, within a cloud network.

According to a second aspect, a SOC based board design is integrated within an integrated surgical device. The SOC based board design comprises one or more primary connectivity ports fabricated over the SOC based board design. The one or more primary connectivity ports establishes connection with one or more energy instruments. Herein, the one or more primary connectivity ports are fabricated to establish high speed connectivity with input devices including the one or more energy instruments. Further, the SOC based board design comprises a processing unit fabricated over the SOC based board design. The processing unit segregates and processes an input data retrieved from the one or more energy instruments into small packets. It can be noted that the input data includes audio, image, video and/or signal data. Further, the SOC based board design comprises a video processing unit (VPU) communicatively coupled to the processing unit. The VPU retrieves the input data and converts into a high-resolution output signal. Further, the SOC based board design comprises one or more secondary connectivity ports fabricated over the SOC based board design. The one or more secondary connectivity ports connects with a display unit to display the high-resolution output signal retrieved from the processing unit and/or VPU. Herein, the one or more secondary connectivity ports are fabricated to establish high speed connectivity with output devices including display unit.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a front view of an integrated surgical device, in accordance with a present embodiment;

FIG. 2 illustrates a display integrated with the integrated surgical device, in accordance with the present embodiment;

FIG. 3 illustrates a back view of the integrated surgical device, in accordance with the present embodiment;

FIG. 4 illustrates a surgical integrated assistance system, in accordance with the present embodiment;

FIG. 5 illustrates a block diagram showing an intra-operation augmented reality and navigation, in accordance with the present embodiment;

FIG. 6 illustrates a motherboard-based design of the integrated surgical device, in accordance with the present embodiment;

FIG. 7 illustrates a cart layout of the surgical integrated assistance system, in accordance with the present embodiment; and

FIGS. 8A and 8B illustrate a System on chip (SOC) based board design architecture of the integrated surgical device, in accordance with the present embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

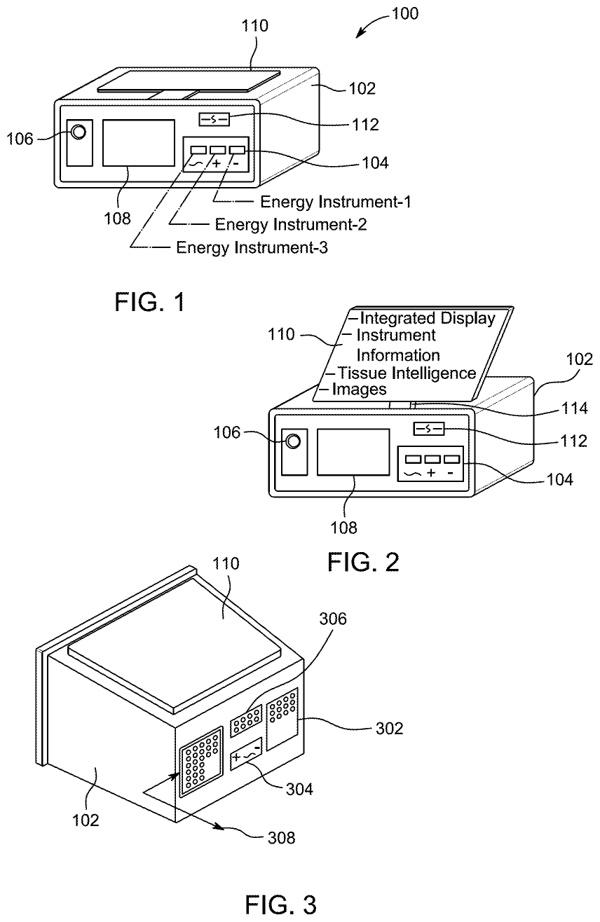

FIG. 1 illustrates a front view of an integrated surgical device 100, according to an embodiment. FIG. 1 is described in conjunction with FIGS. 2-3.

The integrated surgical device 100 may comprise a housing 102 with one or more ports 104, an imaging scope connection 106, a display control unit 108, a display unit 110, a light/sensory source indicator 112, and an artificial intelligence and machine learning (AI/ML) enabled module (not shown). The housing 102 may be a central station or base station for each device or equipment or instrument connected to it. The one or more ports 104 are configured for coupling the integrated surgical device 100 with one or more energy instruments (Energy Instrument-1, Energy Instrument-2, Energy Instrument-3), as shown in FIG. 1. In one embodiment, the one or more energy instruments may include, but are not limited to, bipolar and advanced bipolar shears, monopolar shear, ultrasonic shear, microwave ablation devices, laser ablation devices, laparoscopic devices, robotics control unit, and endoscope. In one embodiment, the imaging scope connection 106, the display control unit 108 and the light/sensory source indicator 112 may be any type of mechanical or electrical connection.

In one embodiment, the integrated surgical device 100 may comprise an energy instrument control module (not shown) configured to control a plurality of parameters of the one or more energy instruments using multiple control means. It may be noted that the energy instrument control module may include selectable options that correspond to, or when selected, execute control functions of the one or more energy instruments. In one embodiment, a user may control the one or more energy instruments by manipulating control means. The control means may include selecting speed of drill, length of rod, power supply of current or voltage etc. The energy instrument control module may include control functionality, such as, buttons, a ball, a foot pedal, and a wheel, permitting navigation through the options of the one or more energy instruments. Thus, the energy instrument control module may have a simplified layout, or a reduced functionality set when compared to a virtual device. For example, the energy instrument control module is limited to navigational control(s) and a selection button, thereby permitting the user to navigate the one or more energy instruments and select a virtual control to activate the desired functionality of the one or more energy instruments. It can be noted that the user may be referred to a doctor, a specialist, an operator, or a lab technician.

Further, the imaging scope connection 106 may be configured for detecting image information within a patient's body. The imaging scope connection 106 may be configured to input at least one optical imaging or an ultrasound imaging instruments. The at least one optical imaging or the ultrasound imaging instruments, may comprise a visual head member (not shown) and an elongated connector (not shown) having a handheld operation portion, an insertion portion, and a first contact element. The at least one optical imaging or the ultrasound imaging instruments, establishes a connection with the housing 102 when the elongated connector is plugged into the imaging scope connection 106.

Further, the display control unit 108 may be capable of driving the one or more energy instruments that are connected to the housing 102. The display control unit 108 may communicate with the housing 102, which in turn may communicate with the one or more energy instruments. The display control unit 108 may send and receive signals to and from the housing 102 to the one or more energy instruments. In one example, the display control unit 108 sends control commands as positioning signals to the housing 102 when selecting an option from, or otherwise interacting with, the one or more energy instruments projected on the display unit 110. It can be noted that the display control unit 108 may include buttons (up, down, left, and right) that allow the user to scroll around the one or more energy instruments. The up, down, left, and right buttons may allow the user to scroll to the desired selection on the one or more energy instruments. It can be noted that the display control unit 108 need not have buttons and may be any type of device that allows the user to navigate the one or more energy instruments. In one embodiment, the display control unit 108 may be a handheld device incorporating a touch pad and/or track wheel/ball, thereby permitting the user to view the one or more energy instruments on the monitor of the computing touch pad or associated screen. The touch pad or track wheel/ball may allow the user to navigate to the energy device and may allow the user to select the desired controls.

In an embodiment, the display unit 110 may be mounted over the housing 102 via an arm 114. The arm 114 may comprise of a hinge mechanism (not shown) to lift the display unit 110 at an angle from the housing 102. The angle of the display unit 110 may be set by the user at a predefined angle between 0 to 90 degrees. It may be noted that the display unit 110 may be configured to fit over a top surface of the housing 102. The arm 114 may not be visible when the display unit 110 is in a closed position over the top surface of the housing 102. In an embodiment, the display unit 110 may be detachably connected with the arm 114 to allow the display unit 110 to detach or attach from the housing. In another embodiment, the display unit 110 may be wirelessly linked with the housing 102 and may be installed over different locations. It can be noted that a portion of the arm 114 may be visible when foldable the display unit 110 is extended as in FIG. 1. It can also be noted that the arm 114 function as a mechanism that prevents the display unit 110 from falling off.

Further, the display unit 110 is configured to show integrated critical information which may be important to assist the user/doctor/surgeon to optimize the procedure flow and decision-making during surgery. The integrated critical information includes, but not limited to, information related to the one or more energy instruments, such as power level, cutting and sealing time, instrument temperature, resonant frequency for ultrasonic device, tissue characteristic, such as tissue impedance, tissue temperature, and rate of change of these characteristics. It may be noted that the information data is integrated with the image data from endoscope or other image to provide additional insights to user to guide the procedures and avoid potential complication during surgery, therefore reducing the patient risks.

Further, the housing 102 may comprise the light/sensory source indicator 112. The light/sensory source indicator 112 may include a light source (not shown), such as, a Light Emitting Diode (LED) mounted with the imaging scope connection 106 or the one or more energy instruments. The light source is provided directly at a visual head member (not shown) to provide improved illumination capability. In one embodiment, the LED may be white light LEDs or LEDs having narrow spectra around a preferred wavelength. In another embodiment, the light source is positioned at a distance from an objective opening of the imaging scope connection 106. For example, when the light source is positioned from the objective opening, the vision head member is provided with means for collecting, reflecting and/or projecting at least a portion of light created by the light source towards a target of the patient's body. The means for collecting, reflecting and/or projecting may be a reflector (not shown) having a deployable formation. In one embodiment, the reflector may be expandable and/or contractible between a smaller diameter to a larger diameter of for example, an iris design comprising a plurality of rigid or semi-rigid members. Further, the light source may be coupled to a plurality of fiber optics (not shown) provided over and along a length of the vision head member. In one embodiment, the plurality of optical fibers may be positioned over an expandable member thereby allowing projection of light in a conical form. The light source is configured to transmit signal to the light/sensory source indicator 112. In one embodiment, the light/sensory source indicator 112 may have an effective area size equal or larger than the outer diameter of the elongated connector.

FIG. 3 illustrates a back view of the integrated surgical device 100, according to the embodiment. The integrated surgical device 100 comprises an external image input port 302, a power port 304, an external intelligence module port 306 and at least one output port 308 integrated on other side of the housing 102. The external image input port 302 enables to connect the one or more energy instruments with the housing 102. Further, the external image input port 302 may be configured to receive input such as images or sensors data from the one or more energy instruments. Further, the external image input port 302 may be configured to receive at least one optical image or an ultrasound image from imaging devices connected to the integrated surgical device 100. The at least one output port 308 may be an instrument information output port. The instrument information output port may include an input unit data (not shown) and an output interface (not shown). The input unit data and the output interface make it possible to integrate data from other therapeutic, diagnostic, or navigational devices. Further, the external intelligence module port 306 may be configured to connect auxiliary devices and energy devices.

In one exemplary embodiment, through interface connection of the imaging scope connection 106, or additional data interface, an energy instrument sends a triggering signal to the external imaging system and laparoscopic insufflator when the activation button or pedal on the energy instrument is pushed. The linked laparoscopic imaging system is triggered to enhance the imaging processing to reduce the impact from the smoke or mist out of the tissue interaction with the energy instrument. This signal can be also implemented as the on/off signal to automatically control the insufflator to reduce the smoke, mist for better image.

In one embodiment, the integrated surgical device 100 may also be cable of connecting with a human/computer interface, such as, touch screen, mouse, or control wheel. This feature gives users an option to input or label on the images. Therefore, the user may control the one or more energy instrument's function and label the one or more energy instruments and tissue interaction during procedure through the computer interface. It may also take place when the energy device is off to just label the tissue or anatomy during procedure for training, navigation, or data collection purpose.

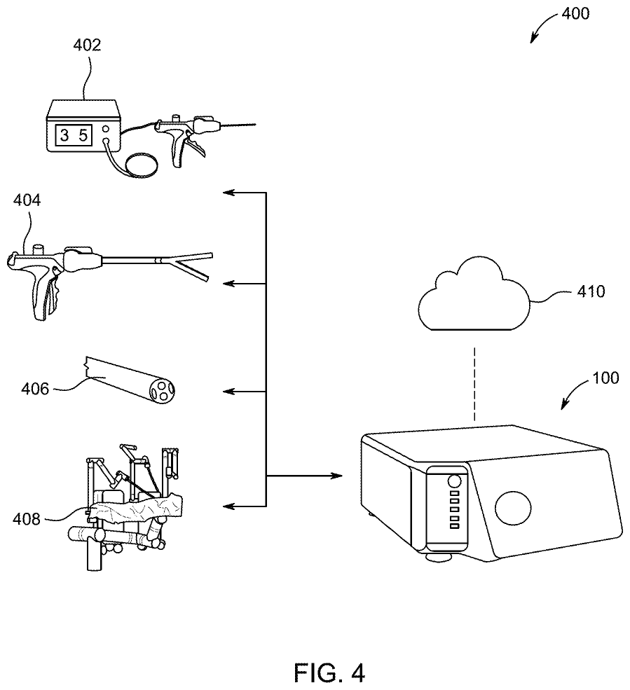

FIG. 4 illustrates a surgical integrated assistance system 400, according to an embodiment.

The surgical integrated assistance system 400 may comprise the integrated surgical device 100. As discussed, the integrated surgical device 100 may be connected to the one or more energy instruments. The one or more energy instruments may include an energy source unit 402, a stapler 404, a laparoscope 406 and a robotic control unit 408. The energy source unit 402 may include bipolar shears, a monopolar shear, and an ultrasonic shear, which may be powered and controlled by the energy source unit 402. The energy source unit 402 comprises an electronic component (not shown) for powering the one or more energy instruments between high voltage and high frequency energy output.

Further, the surgical integrated assistance system 400 may comprise the stapler 404 for automatically stapling the tissues during/after the surgical operations. The laparoscope 406 may include a camera system (not shown), a box of light source (not shown) and a handheld scope (not shown). Further, the robotic control unit 408 that may be configured with a number instrument in a cluster of robotic arms. It will be apparent to a person skilled in the art that the surgical integrated assistance system 400 is not limited to the above-mentioned instruments and there may be any type of energy device/instruments such as an endoscope, a microwave or laser ablation device etc. In an embodiment, the surgical integrated assistance system 400 may be coupled to a cloud network 410 for exchanging information related to the one or more energy instruments and vice versa.

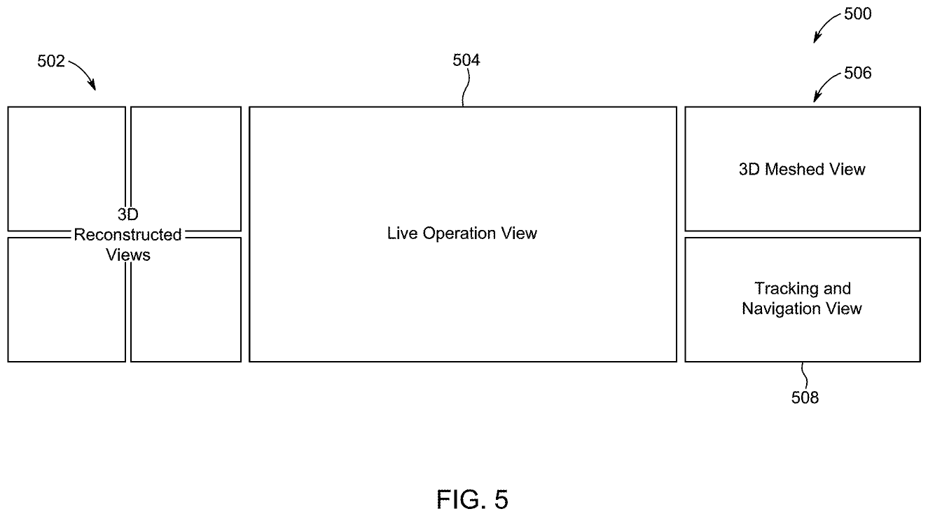

The surgical integrated assistance system 400 may process the information in real time by inferencing of a machine learning model. It may be noted that the machine learning model may be trained by collecting operation data from the one or more energy instruments. Further, the surgical integrated assistance system 400 may provide a consolidated visualization output from the one or more energy instruments. Further, each of the one or more energy instruments may have a unique intelligence feature illustration, as shown in FIG. 5.

FIG. 5 illustrates a block diagram showing exemplary images 500 of an intra-operation augmented reality and navigation. The exemplary images 500 show a three-dimensional (3D) reconstructed views 502. The 3D reconstructed views 502 may be a 3D reconstruction of pre-operation image showing Computer Tomography (CT) scan or Magnetic Resonance Imaging (MRI) images. Further, a live operation view 504 is shown. The live operation view 504 may include live streaming or live images of a surgical procedure, such as laparoscopy. Further, a 3D meshed view 506 is displayed. The 3D meshed view 506 may be a 3D view of a patient's organs. Further, a tracking and navigation view 508 is displayed. The tracking and navigation view 508 may be a real-time tissue tracking and navigation. The real-time tissue tracking, and navigation may include an augmented reality for lesion and nodule, localization, and navigation.

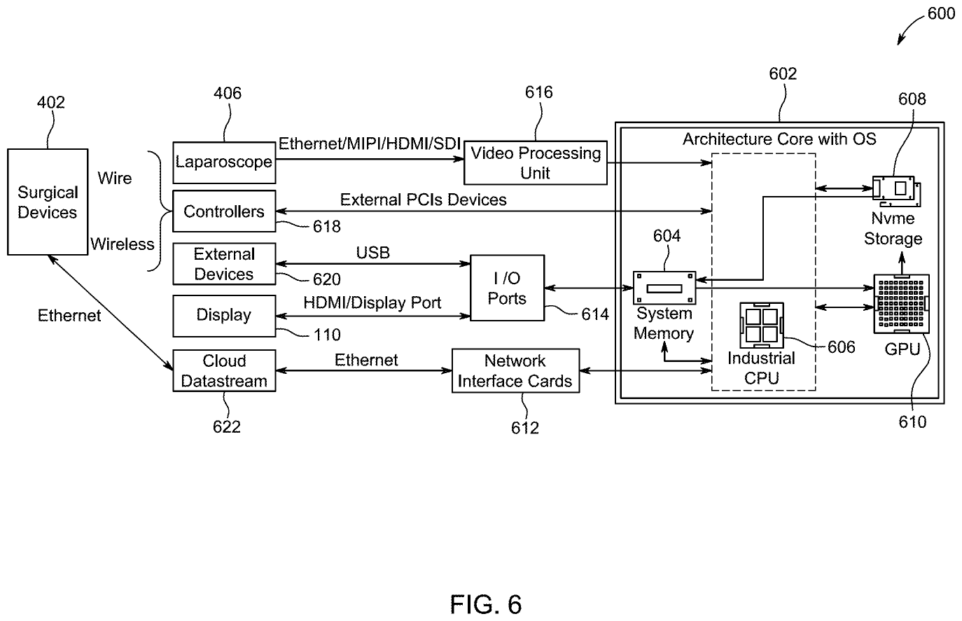

FIG. 6 illustrates a motherboard-based design of the integrated surgical device 100, in accordance with the present embodiment. The motherboard-based design may correspond to an artificial intelligence and machine learning (AI/ML) enabled module 600. The AI/ML enabled module 600 comprises the energy source unit 402, a motherboard 602 having a system memory 604, an industrial central processing unit (CPU) 606, a Nonvolatile memory express (Nvme) storage 608, and a graphical processing unit (GPU) 610. Further, the AI/ML enabled module 600 comprises network interface cards 612, I/O ports 614 and a video processing unit 616. Further, the energy source unit 402 is configured to provide input and power that may connect through USB or wirelessly, to the laparoscope 406, the one or more energy instruments, controllers 618, external devices 620, a cloud data stream 622, and the display unit 110. Further, the laparoscope 406, the controllers 618, the display unit 110 are coupled to different elements of the motherboard 602. In one embodiment, the external devices 620 may include the one or energy instruments connected to the integrated surgical device 100 via the one or more ports 104, and the imaging devices connected via the imaging scope connection 106.

Further, the system memory 604 may be coupled to the industrial CPU 606. The system memory 604 may include suitable logic, circuitry, and/or interfaces that may be configured to store a machine code and/or a computer program with at least one code section executable by a processor. Examples of implementation of the memory may include, but are not limited to, Random Access Memory (RAM), Read Only Memory (ROM), Hard Disk Drive (HDD), and/or a Secure Digital (SD) card.

Further, the industrial CPU 606 is coupled to system memory 604, the network interface cards 612, the GPU and the Nvme storage 608. Further, the industrial CPU 606 comprises a processor that may include suitable logic, circuitry, interfaces, and/or code that may be configured to execute a set of instructions stored in the system memory 604. The processor may be configured to generate display image or video. The processor may be further configured to receive the energy source unit 402 data via the cloud data stream 622. Examples of the processor may be an X86-based processor, a Reduced Instruction Set Computing (RISC) processor, an Application-Specific Integrated Circuit (ASIC) processor, a Complex Instruction Set Computing (CISC) processor, and/or other processors.

In an implementation, the Nvme storage 608 may be integrated with the industrial CPU 606. The Nvme storage 608 may be configured to store the information of the input data received from the energy source unit 402. The Nvme storage 608 may be configured to store the details of the input signal received from via the industrial CPU 606. A person of ordinary skill in the art will appreciate that the data stored in the databases described above may be stored in a structured or an unstructured data format. Examples of implementation of the Nvme storage 608 described above may include, but are not limited to, secure databases such as Amazon Web Services Cloud (AWS®), Microsoft Azure®, Cosmos DB®, Oracle® Database, Sybase®, MySQL®, Microsoft Access®, Microsoft SQL Server®, FileMaker Pro™, and dBASE®. A person of ordinary skill in the art will appreciate that in an alternate implementation, the databases described above may be implemented as an entity that is separate from the Nvme storage 608, without limiting the scope of this disclosure. Further, the GPU 610 may be coupled to the Nvme storage 608 and the industrial CPU 606 and is configured to provide additional computational power required for the generation of display data. It may be noted that the GPU coupled to storage reduce a buffer talk.

The I/O ports 614 coupled to the motherboard 602 may include suitable logic, circuitry, interfaces, and/or code that may be configured to receive signal from the energy device. The I/O ports 614 may include various input and output devices that may be configured to facilitate the communication between the external device and the display unit 110. In one embodiment, the display unit 110 may be referred as a foldable additional display. It is such one or more electronic devices that, in conjunction with the I/O ports 614, may be configured to present display data one or more interfaces on the additional display in an instance. Examples of the display screen may include, but are not limited to, Liquid Crystal Display (LCD) display, Light Emitting Diode (LED) display, Organic LED (OLED) display technology, and/or the like.

The network interface cards 612 may include suitable logic, circuitry, interfaces, and/or code that may be configured to facilitate communication among the plurality of energy devices on energy source unit via the cloud data stream 622. The network interface cards 612 may be implemented based on known technologies to support wired or wireless communication. The network interface cards 612 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, and/or a local buffer. The network interface cards 612 may communicate via wireless communication with networks, such as the Internet, an Intranet and/or a wireless network, such as a cellular telephone network, a wireless local area network (LAN) and/or a metropolitan area network (MAN). The wireless communication may use any of a plurality of communication standards, protocols and technologies, such as Global System for Mobile Communications (GSM®), Enhanced Data GSM Environment (EDGE®), wideband code division multiple access (W-CDMA®), code division multiple access (CDMA®), Long Term Evolution (LTE®), time division multiple access (TDMA), Bluetooth®, Wireless Fidelity (Wi-Fi®) (such as IEEE® 802.11a, IEEE® 802.11b, IEEE 802.11g and/or IEEE® 802.11n), voice over Internet Protocol (VoIP®), Wi-MAX®, a protocol for email, instant messaging, and/or Short Message Service (SMS).

The video processing unit 616 may be configured to process low quality images and or video footages and convert into high quality footages. Further, the industrial CPU 606 in conjunction with the video processing unit 616, may be configured to generate a display the multimedia content on the display unit 110. It can be noted that the video processing unit 616 may be referred to as a video recorder.

Further, the AI/ML enabled module 600 is configured to acquire data from energy source unit 402. Further, the AI/ML enabled module 600 computes intra operation in real time processing of surgical video, thus improve localization and surgical navigation. Further, the AI/ML enabled module 600 is configured to upload data to the cloud network 410. Further, the AI/ML enabled module 600 generates 3D reconstruction of the multi organ model using pre-operation MRI or CT images as the input. The AI/ML enabled module 600 is configured to generate a real time inference with the live video feed from Laparoscope to highlight the location of the lesions, nodules, or lymph nodes, along with outline of key nerve, vein, and artery. A navigated path to operate on the lesion or nodules is illustrated.

Further, the AI/ML enabled module 600 may be configured to train a deep learning model. In an embodiment, the deep learning model at the initial steps is trained using the information collected by the one or more energy instruments. It can be noted that the deep learning model is configured to process input data from the one or more energy instruments for predicting output conditions of the one or more energy instruments.

In an embodiment, the AI/ML enabled module 600 is linked with the deep learning model to process the input data from one or more sensors integrated with the one or more energy instruments. The AI/ML enabled module 600 may be connected to the housing 102 to form the surgical integrated assistance system 400. The AI/ML enabled module 600 is trained to enhance the imaging processing to reduce the impact from the parameters such as the smoke or mist out of the tissue interaction with the one or more energy instruments. Further, the AI/ML enabled module 600 is trained to implement the on/off signal to one or more energy instruments for automatically control for example the insufflator to reduce the smoke, mist for better image. Further, the AI/ML enabled module 600 generates multiple inputs having multiple possibilities of accurate outputs, using a qubit calculation. In one embodiment, the AI/ML enabled module 600 is trained using deep learning models, with one layer of input data as an input to another layer, to generate a predicted output.

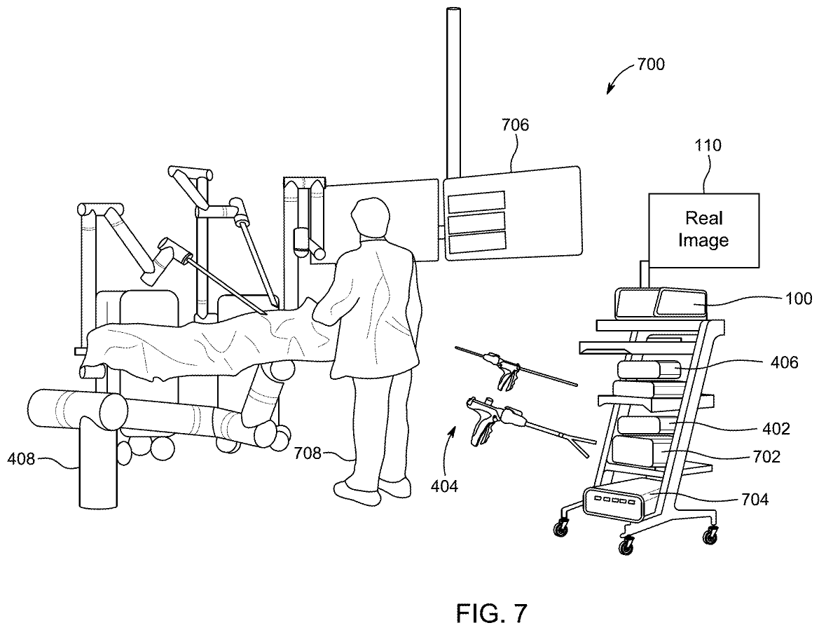

FIG. 7 illustrates a cart layout 700 of the surgical integrated assistance system 400, according to an embodiment. The cart layout comprises the one or more energy instruments as shown in FIG. 4. The one or more energy instruments are connected to the AI/ML enabled module 600 sends digital control signal to the energy instrument control module of the one or more energy instruments, via a Peripheral Component Interconnect Express (PCIE). The one or more energy instruments may include the laparoscope 406, the energy source unit 402, the robotic control unit 408, a robotic controller 702, an insufflator 704. Further, the cart layout 700 may include a display screen 706 to monitor parameters of the one or more energy instruments while performing the surgical operation. Therefore, a surgeon 708 may access the required data from the AI/ML enabled module 600. In one embodiment, the surgeon 708 may provide commands to the one or more energy instruments while receiving suggestions from the AI/ML enabled module 600. It can be noted that the surgeon 708 may be referred to as a doctor or a specialist, an operator, or a lab technician.

The AI/ML enabled module 600 is configured to generate the 3D reconstruction of the multi organ model using the pre-operation MRI or CT Scan images as the reference points. Further, AI/ML enabled module 600 is configured to collect real time inference with live video feed from the one or more energy instruments to highlight location of diagnosis. In an embodiment, the AI/ML enabled module 600 is configured to calculate operation curve of each of the one or more energy instruments to display on the display unit 110. The curve provides information related to progress of the diagnosis and display operation status reminder of the one or more energy instruments for surgeon's reference.

In an embodiment the AI/ML enabled module 600 is configured to retrieve real-time streaming video from each of the one or more energy instruments in operation. Further, the AI/ML enabled module 600 is configured to compute a machine learning inference for lesion localization and surgical navigation. Further, the AI/ML enabled module 600 is configured to process intra-operation real-time surgical video and upload and store the surgical video and data related to each of the one or more energy instruments in operation within the cloud network 410.

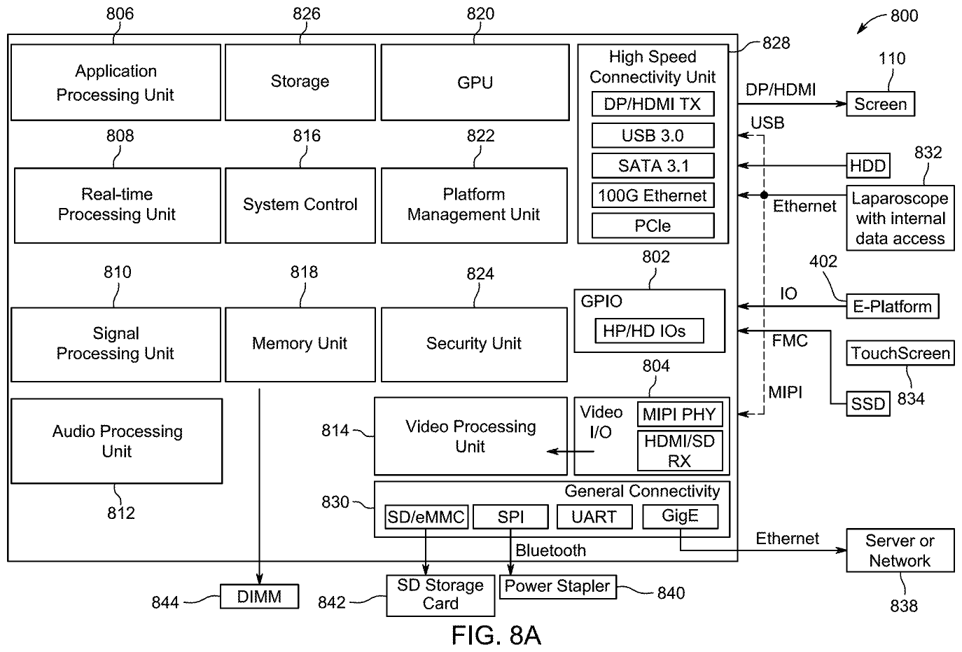

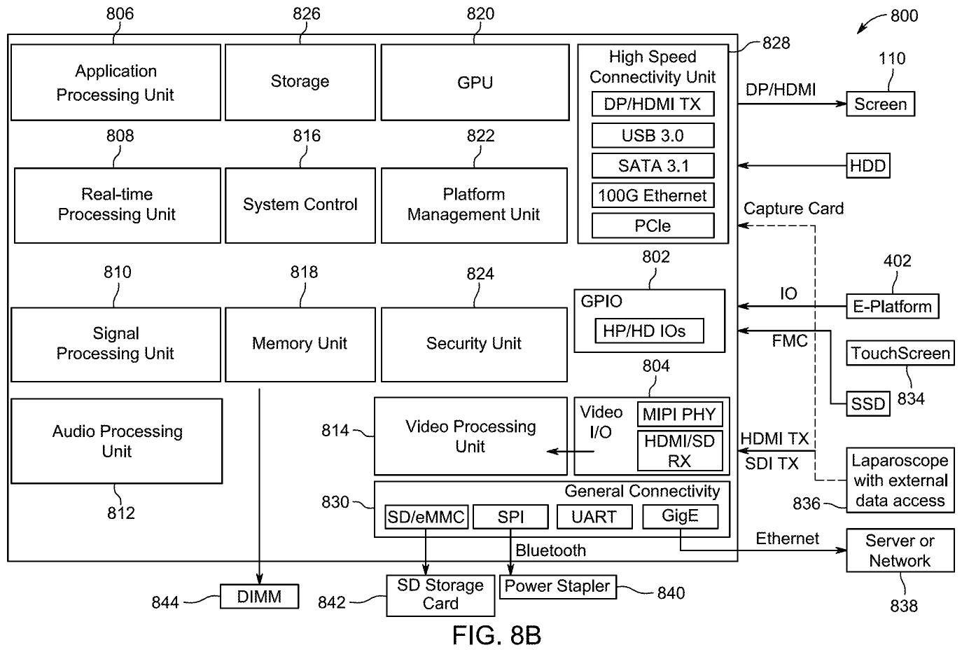

FIGS. 8A and 8B illustrates a System on chip (SOC) based board design 800 architecture of the integrated surgical device, according to an embodiment.

The SOC based board design 800 may be referred as a system-on-chip (SoC) design architecture. The SOC based board design 800 is capable of hardware-oriented user programming that improves the stability of the surgical integrated assistance system 400, the coding level of the driver and the interference of one or more energy instruments which may cause problems such as delays. Further, the SOC based board design 800 may be configured to provide full user customization, including the form factor. The SOC based board design 800 comprises one or more primary connectivity ports, a processing unit, and one or more secondary connectivity ports.

Further, the one or more primary connectivity ports includes a general-purpose input/output (GPIO) 802, a Video Input/Output 804. The GPIO 802 and the Video Input/Output 804 are used for connecting the one or more energy instruments to execute the surgical activities. Further, the SOC based board design 800 includes the processing unit configured to receive and process input data from the one or more energy instruments. The processing unit comprises of an application processing unit 806, a real-time processing unit 808, a signal processing unit 810 and an audio processing unit 812. Further, the application processing unit 806 provides a general-purpose computing in a standard programming environment based on the SOC based board design 800.

In one embodiment, the processing unit segregates the input data received from the one or more energy instruments into small packets and process the input data independently to eliminate lag, delay, or interferences between the input data of the one or more energy devices. Further, the real-time processing unit 808 is configured to execute real-time processing in which the surgical integrated assistance system 400 may input rapidly changing data received from the GPIO 802 and Video Input/Output 804 and then provide output instantaneously so that the change over time may be seen very quickly. It can be noted that the real-time processing unit 808 performs an instantaneous processing of data, when data input requests need to be dealt with quickly. Further, the signal processing unit 810 is configured to manipulate information content in signals to facilitate automatic speech recognition (ASR). It can be noted that the ASR helps to extract information from the speech signals and then translate the extracted information into recognizable words. Further, the audio processing unit 812 is configured to convert between analog and digital formats, to cut or boost selected frequency ranges, to remove unwanted noise, to add effects and to obtain many other desired results.

Further, the SOC based board design 800 is configured with a video processing unit (VPU) 814 to retrieve the input data and convert into a high-resolution output signal. The VPU 814 reduces the need for external capture card system. In one exemplary embodiment, the VPU 814 may have a programmable logic as H.265 encoder and decoder to input and output 4K60 videos. It can be noted that the SOC based board design 800 is a printed circuit board (PCB) design, and it is feasible to combine the VPU 814 with different Video Input/Output 804, such as, high-definition multimedia interface (HDMI), serial digital interface (SDI), etc.

Further, the one or more secondary connectivity ports are fabricated over the SOC based board design 800. The one or more secondary connectivity ports connects with a display unit to display the high-resolution output signal retrieved from the processing unit and/or VPU 814. Further, the SOC based board design 800 includes a system control 816, a memory unit 818, a graphical processing unit (GPU) 820, a platform management unit 822, a security unit 824 and a storage 826. Herein, the storage 826 is configured to save and/or input data related to surgical procedures or for the one or more energy instruments. The system control 816, the memory unit 818, the GPU 820, the platform management unit 822 and the security unit 824 works in synchronization with the processing unit. The one or more secondary connectivity ports includes a high-speed connectivity unit 828 and a general connectivity 830.

Further, the SOC based board design 800 may be linked to the display unit 110, via the high-speed connectivity unit 828. It can be noted that the display unit 110 may be referred to as a screen, as shown in FIGS. 8A-8B. In one embodiment, the high-speed connectivity 828 may allow connectivity with multiple devices used in minimally invasive procedure. The multiple devices may include the laparoscope 406, the stapling device 404, and the energy device 402. Further, the high-speed connectivity unit 828 may allow connection of the SOC based board design 800 with a laparoscope with internal data access 832 using capture cards over Ethernet. In one embodiment, the laparoscope with internal data access 832 refers to laparoscope that shares data architecture, which allow access of the image data at various breakout point of data collecting and transfer path. It can be noted that the laparoscope with internal data access 832 may store information in a hard disk drive (HDD) format. Herein, the laparoscope with internal data access 832 may alternatively link with a USB 3.0 to the high-speed connectivity unit 828. Further, the laparoscope with internal data access 832 may be connected to 100G Ethernet via Ethernet and to MIPI PHY as shown in FIG. 8A.

Further, the high-speed connectivity unit 828 may allow connection of the SOC based board design 800 with a laparoscope with external data access 836 using capture cards over Ethernet. In one embodiment, the laparoscope with external data access 836 refers to a third party laparoscope and the access of the image data can only be through the capture cards. The laparoscope with external data access 836 may be alternatively linked to PCLe via a capture card and the video input/output unit 804 through HDMI TX or SDI TX ports. It can be noted that the laparoscope with internal data access 832 and the laparoscope with external data access 836 may be referred as an internal laparoscopy and external laparoscopy. Further, the GPIO 802 may be linked with a touchscreen 834 for receiving instructions or commands from the surgeon 708 related to various surgical activities. It can be noted that the GPIO 802 may store and execute instructions in a solid-state drive (SSD) format.

Further, the general connectivity 830 may comprise a Gigabit Ethernet (GigE), an embedded multimedia card (SD/eMMC), a serial peripheral interface (SPI), and a universal asynchronous receiver-transmitter (UART). Further, GigE may be a tethered protocol for data transfer based on a widespread Ethernet standard and may be connected to a server or network 838. Further, SPI may be connected to a power stapler 840 and SD/eMMC may be connected to an SD storage card 842, via a Bluetooth connection.

Further, the memory unit 818 may be connected to a dual in-line memory module (DIMM) 844 for transfer of data related to each of the one or more instruments.

In an embodiment, the processing unit may directly program and process the input data, thus reducing the latency in reading and sending data without changing driver or operating system. For example, the surgeon 708 may directly process any port (e.g., USB) data directly without accessing an operating system (OS) and firmware of motherboard. The hardware property of SoC has some key features to improve performance. The real-time processing unit 808 may accelerate the machine learning algorithm. One of the features is that the processing unit requires the less power so that it will not have a big thermal mitigation problem. The processing unit also has great hardware capacity extendibility. In one embodiment, the processing unit may have the PCIE slot to connect additional high-speed component. In another embodiment, the processing unit may have a SATA port to support the large storage.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include random access memory (RAM), read-only memory (ROM), volatile memory, nonvolatile memory, hard drives, CD ROMs, DVDs, flash drives, disks, and any other known physical storage media.

The features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Features that are described and/or illustrated with respect to one embodiment may be used in the same way or in a similar way in one or more other embodiments and/or in combination with or instead of the features of the other embodiments.

While the preferred embodiment of the present invention has been illustrated and described, as noted above, many changes can be made without departing from the spirit and scope of the invention. For example, aspects of the present invention may be adopted on alternative operating systems. Accordingly, the scope of the invention is not limited by the disclosure of the preferred embodiment. Instead, the invention should be determined entirely by reference to the claims that follow.

### LIST OF ELEMENTS

### A Surgical Integrated Assistance System

100 Integrated Surgical Device
102 Housing
104 One or more Ports
106 Imaging Scope Connection
108 Display Control Unit
110 Display Unit
112 Light/Sensory Source
114 Arm
302 External Image Input Port
304 Power Port
306 External Intelligence Module Port
308 At least one Output Port
400 Surgical Integrated Assistance System
402 Energy source unit
404 Stapler
406 Laparoscope
408 Robotic Control Unit
410 Cloud Server
500 Screen mock-up of an Intra-Operation Augmented Reality and Navigation
600 Artificial Intelligence and Machine Learning (AI/ML) enabled module
602 Motherboard
604 System Memory
606 Industrial Central Processing Unit (CPU)
608 Storage
610 Graphical Processing Unit (GPU)
612 Network Interface Cards
614 I/O Ports
616 Video processing unit
618 Controllers
620 External Devices
622 Cloud Data Stream
700 Cart Layout
702 Robotic Controller
704 Insufflator
706 Display Screen
708 Surgeon
800 SOC based board design
802 General-Purpose Input/Output (GPIO)
804 Video Input/Output
806 Application Processing Unit
808 Real-Time Processing Unit
810 Signal Processing Unit
812 Audio Processing Unit
814 Video Processing Unit (VPU)
816 System Control
818 Memory Unit
820 Graphical Processing Unit (GPU)
822 Platform Management Unit
824 Security Unit
826 Storage Unit
828 High-Speed Connectivity Unit
830 General Connectivity
832 Laparoscope with Internal Data Access
834 Touchscreen
836 Laparoscope with External Data Access
838 Server or Network
840 Power Stapler
842 SD Storage Card
844 Dual In-Line Memory Module (DIMM)

What is claimed is:

1. A surgical integrated assistance system comprising:
an integrated surgical device having:
a housing having one or more ports integrated at one side of the housing, wherein the one or more ports are configured for coupling the integrated surgical device with one or more energy instruments;
a display control unit disposed on the side of the housing, and configured to control operating mechanism of each of the one or more energy instruments; and
an artificial intelligence and machine learning (AI/ML) enabled module integrated within the housing, to automatically optimize multiple parameters of the one or more energy instruments.

2. The surgical integrated assistance system of claim 1, wherein the one or more energy instruments include at least one of bipolar and advanced bipolar shears, monopolar shear, ultrasonic shear, microwave ablation devices, laser ablation devices, laparoscopic devices, robotics control unit, and endoscope.

3. The surgical integrated assistance system of claim 1, wherein each of the one or more energy instruments are controlled between high voltage and high frequency energy output of the integrated surgical device.

4. The surgical integrated assistance system of claim 1, wherein a display unit is detachably mounted onto the housing and configured to provide a consolidated output related to the one or more energy instruments.

5. The surgical integrated assistance system of claim 1, wherein the integrated surgical device having at least one external image input port, at least one output port, a power port, and at least one external intelligence module port, on the other side of the housing.

6. The surgical integrated assistance system of claim 5, wherein the at least one output port is configured for transmitting an output energy device data, a surgical instrument image data, an instrument information and tissue intelligence.

7. The surgical integrated assistance system of claim 4, wherein the display unit is configured to display integrated display instrument information, tissue intelligence information, and at least one optical image or an ultrasound image.

8. The surgical integrated assistance system of claim 1, wherein the AI/ML enabled module is configured for data acquisition, computation, automation, multi-modality devices connection and data storage.

9. The surgical integrated assistance system of claim 1, wherein the AI/ML enabled module is communicatively coupled to a cloud network for training models of the AI/ML enabled module and to collect real-time information related to the one or more energy instruments.

10. The surgical integrated assistance system of claim 1, wherein the AI/ML enabled module is configured to:
generate a three-dimensional (3D) reconstruction of a multi-organ model using a pre-operation magnetic resonance imaging (MRI) or computed tomography (CT) scan images as reference points;
collect real time inference with live video feed from the one or more energy instruments to highlight location of diagnosis;
calculate operation curve of each of the one or more energy instruments to display onto the display unit, wherein the operation curve provides information related to progress of the diagnosis; and
display an operation status reminder of the one or more energy instruments for surgeon's reference.

11. The surgical integrated assistance system of claim 10, wherein the AI/ML enabled module is configured to:
retrieve real-time streaming video from each of the one or more energy instruments in operation;
compute a machine learning inference for lesion localization and surgical navigation;
process intra-operation real-time surgical video; and
upload and store the surgical video and data related to each of the one or more energy instruments, employed in an operation, within the cloud network.

12. A system on chip (SOC) based board design integrated within an integrated surgical device, wherein the SOC based board design comprising:
one or more primary connectivity ports fabricated over the SOC based board design, wherein the one or more primary connectivity ports establish connection with one or more energy instruments;
a processing unit fabricated over the SOC based board design, and configured to segregate and process an input data retrieved from the one or more energy instruments into small packets;
a video processing unit (VPU) communicatively coupled to the processing unit, and configured to retrieve the input data and convert into a high-resolution output signal; and
one or more secondary connectivity ports fabricated over the SOC based board design and connect with a display unit to display the high-resolution output signal retrieved from the processing unit and/or VPU.

13. The SOC based board design of claim 12, wherein the processing unit is configured to provide stability while processing the input data by eliminating possible interference and delays caused due to the connected one or more energy instruments.

14. The SOC based board design of claim 12, wherein the processing unit segregates and processes the input data into small packets to eliminate delay and interferences of the input data between the one or more energy instruments.

15. The SOC based board design of claim 12, wherein the processing unit is configured to provide customization to the operator to enhance usability and form factor during conducting an operation.

16. The SOC based board design of claim 12, wherein the input data includes audio, image, video and/or signal data.

17. The SOC based board design of claim 12, wherein the VPU is configured to input and output 4K 60 Hz videos.

18. The SOC based board design of claim 12, wherein the one or more primary connectivity ports are fabricated to establish high speed connectivity with the one or more energy instruments.

19. The SOC based board design of claim 12, wherein the one or more secondary connectivity ports are fabricated to establish high speed connectivity with the display unit.
